(19) United States
(12) Patent Application Publication
(10) Pub. No.: US 2022/0273365 A1
(43) Pub. Date: Sep. 1, 2022

(54) Nerve Repair Using Laser Sealing

(71) Applicants: Arizona Board of Regents on behalf of Arizona State University, Scottsdale, AZ (US); MAYO FOUNDATION FOR MEDICAL EDUCATION AND RESEARCH, Rochester, MN (US)

(72) Inventors: Kaushal Rege, Chandler, AZ (US); Inam Ridha, Tempe, AZ (US); Russell Urie, Tempe, AZ (US); Pelagia Kouloumberis, Paradise Valley, AZ (US); Shelley Noland, Scottsdale, AZ (US)

(21) Appl. No.: 17/637,484

(22) PCT Filed: Aug. 27, 2020
(86) PCT No.: PCT/US2020/048225

§ 371 Date: Feb. 23, 2022

Related U.S. Application Data
(60) Provisional application No. 62/894,248, filed on Aug. 30, 2019.

(51) Int. Cl. A61B 18/20; A61L 24/02; A61L 24/00; A61L 27/04; A61L 27/50; A61L 27/54; A61L 27/58

(52) U.S. Cl. CPC A61B 18/20; A61L 24/02; A61L 24/001; A61L 24/0042; A61L 24/0015; A61L 27/047; A61L 27/50; A61L 27/54; A61L 27/58; A61B 2018/00434

(57) ABSTRACT

The present invention provides a method of nerve repair using localized delivery of heat. The method involves localized induction of hyperthermia for end-to-end attachment of severed peripheral nerves by delivering stimulus responsive materials and exposing them to an excitation source under conditions wherein they emit heat. The generation of heat effects the joining of the nerve ends.

### CROSS REFERENCE TO RELATED APPLICATIONS

This application claims priority to U.S. Provisional Application No. 62/894,248, filed Aug. 30, 2019, which is hereby incorporated by reference herein in its entirety.

### STATEMENT REGARDING FEDERALLY SPONSORED RESEARCH OR DEVELOPMENT

This invention was made with government support under Grant No. R01 EB020690 awarded by National Institute of Health. The government has certain rights in the invention.

### BACKGROUND OF THE INVENTION

Nerve damage affects thousands of patients every year as a result of trauma such as military activities, sports injuries, surgical procedures and neuropathies; each year at least 200,000 trauma-related nerve injuries occur in the US. Severe peripheral nerve injuries have devastating impact on a patient's quality of life. Poor management of nerve injuries is associated with muscle atrophy and can lead to painful neuroma when severed axons are unable to reestablish continuity with the distal nerve. Although nerves have the potential to regenerate after injury, this ability is strictly dependent upon the regenerating nerve fibers making appropriate contact with the severed nerve segment. Thus, surgical intervention is typically needed in many cases. If the nerve fibers are detached, reattachment is typically accomplished by end-to-end anastomosis or by the insertion of nerve grafts.

Despite the long history and improvements in microsurgical research and surgeries, peripheral nerve repair remains a challenge. Using sutures to hold nerve fibers together puts significant stress on nerve and blood flow and can lead to hindrance in nerve growth. In addition, existing tissue adhesives demonstrate sub-optimal strength in this highly dynamic environment. Adhesives typically suffer from low adhesion, low elasticity, and low stiffness compared to suture materials.

Thus, there is a need in the art for compositions and methods to facilitate faster repair and regeneration. The present invention meets this need.

### SUMMARY OF THE INVENTION

In one aspect, the present invention provides a method for welding tissue wounds in a subject, wherein the method comprises the steps of: providing a scaffold, wherein the scaffold comprises a base structural material and at least one stimulus responsive component; aligning a first edge of the wound with a second edge of the wound; placing the scaffold over or in between the first edge of the wound and the second edge of the wound; and exposing the scaffold to an internal or external energy source, wherein the stimulus responsive component absorbs the energy and subsequently generate heat and causes the first edge of the wound and the second edge of the wound to adhere to each other and/or to the scaffolds.

In one embodiment, the structural material is selected from the group consisting of: a natural polymer, a synthetic polymer and a combination thereof. In one embodiment, the scaffold is selected from the group consisting of: a polymeric matrix, a hydrogel, a film, an electrospun scaffold, a tubular conduit and combinations thereof. In one embodiment, the structural material is chemically modified to facilitate adhesion of the scaffold to the tissue. In one embodiment, the stimulus responsive material is selected from the group consisting of: a photoresponsive material, a magnetic responsive material, an electrically responsive material, a chemically responsive material and combinations thereof. In one embodiment, the stimulus responsive material is a photoresponsive material. In one embodiment, the stimulus responsive material is in particle form. In one embodiment, the stimulus responsive material is selected from a group consisting of gold nanorods, gold nanostars, gold nanoparticles, gold nanospheres, gold nanostars, indocyanin green, neodymium-doped nanoparticles, carbon nanotubes, organic nanoparticles, alumina nanoparticles, copper nanoparticles or near-infrared absorbing dyes, silver nanoparticles, silver nanoplats/prisms, and combinations thereof. In one embodiment, the photoresponsive material is stimulated with a laser. In one embodiment, the laser wavelength is in a range of between 800 nm to about 2700 nm. In one embodiment, the laser is delivered in pulse mode wherein a series of short pulses are applied. In one embodiment, the laser is delivered in a continuous mode. In one embodiment, the scaffold further comprises an active agent selected from the group consisting of: an anti-inflammatory, a wound healing agent, a growth factor and combinations thereof. In one embodiment, the structural material is biodegradable. In one embodiment, the tissue is selected from a group consisting of: skin, mucosal tissue, bone, blood vessels, neural tissue, hepatic tissue, pancreatic tissue, splenic tissue, renal tissue, bronchial tissue, tissues of the respiratory tract, tissues of the urinary tract, tissues of the gastrointestinal tract, tissues of the gynecologic tract and combinations thereof. In one embodiment, the tissue is a neural tissue.

In one aspect, the present invention provides a composition for welding tissue wounds in a subject, wherein the composition comprises a scaffold, wherein the scaffold comprises a base structural material and at least one stimulus responsive component. In one embodiment, the scaffold is selected from the group consisting of: a polymeric matrix, a hydrogel, a film, an electrospun scaffold, a tubular conduit and combinations thereof. In one embodiment, the structural material is selected from the group consisting of: a natural polymer, a synthetic polymer, and combinations thereof. In one embodiment, the stimulus responsive material is selected from the group consisting of: a photoresponsive material, a magnetic responsive material, an electrically responsive material, a chemically responsive material and combinations thereof.

### BRIEF DESCRIPTION OF THE DRAWINGS

The following detailed description of invention will be better understood when read in conjunction with the appended drawings. It should be understood, however, that the invention is not limited to the precise arrangements and instrumentalities of the embodiments shown in the drawings.

FIG. 1 is a flowchart depicting an exemplary method of repairing nerve injury using laser sealing.

FIG. 2 depicts photothermal response for Silk, Chitosan-GA, gAlginate, and gCellulose films to the mid-infrared laser light (6.5 μm laser wavelength) with the power density of 1.19 W/cm². All the sealant films were in the circular shape with the diameter of 1 cm.

FIG. 3 depicts temperature of the Chitosan containing gold nanorods (GNRs) upon laser illumination. The film was subjected to the laser beam for 30 seconds and rest for another 30 seconds. The cycle was repeated three times.

FIG. 4 depicts end-to-end anastomosis of rat sciatic nerve ex vivo using chitosan doped with gold nanorods and irradiated with near infrared laser beam at 2 W/cm.

FIG. 5 depicts an exemplary schematic of NILAA mediated sealing and repair of small and large defects in peripheral nerves. NILAA glues and tapes are employed for end-to-end anastomosis of nerves with small (<5 mm) gaps. NILAA tapes are developed for facilitating tissue adhesion of regenerative conduits as a faster suture-less approach. In all cases, hand-held NIR lasers facilitate rapid sealing and clinical translation.

FIG. 6 comprising FIG. 6A through FIG. 6C depicts an exemplary schematic of epinureal suturing of severed nerves. FIG. 6A depicts that NILAA provides epinureal sealing. FIG. 6B depicts that NILAA provides epinureal wrapping. FIG. 6C depicts an exemplary schematic of interdigized NILAA molecules (grey) and epineurium (red) upon sealing.

FIG. 7 comprising FIG. 7A through FIG. 7B depicts a schematic of a developed mathematical model for temperature responses in nerves. FIG. 7A depicts a profile view of the predicted temperature gradient through the x-z plane. The dashed line (-) represents the NILAA (1 mm)-tissue (4 mm) interface, with the area above encompassing the patch and the area below encompassing the tissue. FIG. 7B depicts a prediction of temperature response of porcine intestines compared to experimental data following NIR irradiation of collagen-GNR NILAA. Solid lines are model predictions and points are experimental data. Black: On the surface of the intestine Grey: in the lumen. The NILAA is placed only on the surface.

FIG. 8 comprising FIG. 8A through FIG. 8B depicts remote stimulation of sciatic nerve using cuff electrodes and implanted diodes. FIG. 8A depicts experimental setup to measure EMG from 3 different muscle groups in response to stimulation of the sciatic nerve using a cuff electrode. FIG. 8B depicts a biphasic, rectangular voltage stimulus (250 μsec duration, 10 Hz repetition rate) is applied between rings ‘1’ and ‘9’ spaced 2.7 mm apart in the cuff electrode. Typical EMG responses to voltage stimulation are overlaid (n=10 replicates) showing consistency in the amplitudes and latencies.

### DETAILED DESCRIPTION

The present invention provides compositions and methods for promoting the repair and/or growth of nerve tissue. The compositions and methods of the subject invention can be employed to restore the continuity of nerves interrupted by disease, traumatic events or surgical procedures. The compositions and methods of the subject invention promote repair of nerve tissue by the growth of axons that successfully penetrate damaged nerve tissue or implanted nerve grafts, resulting in greater functional recovery. The method of the subject invention provides the means to align and fix tissue wounds in place, using a scaffold wherein the scaffold is placed over the injury site to join the tissue edges together. The method involves using of a stimulus responsive components in the scaffold, that when exposed to an excitation source, creates localized heating and in return effects tissue repair.

### Definitions

It is to be understood that the figures and descriptions of the present invention have been simplified to illustrate elements that are relevant for a clear understanding of the present invention, while eliminating, for the purpose of clarity, many other elements typically found in the art. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range.

### Method of Use

In one embodiment, the method of the subject invention provides the means to align and fix tissue wounds in place, using a scaffold wherein the scaffold comprises a base structural material and at least one stimulus responsive component.

In one aspect, the method of the subject invention provides the means for precise placement and rapid attachment of a scaffold at an injury site. In one aspect, the invention provides a method of repairing nerve tissue without inducing compression or interfering with cellular and molecular processes in nerve regeneration. In one embodiment, the method of the subject invention includes providing a stronger and more permanent union between the nerve ends. In one embodiment, the method of the subject invention can also reduce fibrotic scarring associated with nerve repair and regeneration. In one aspect, the method of this invention includes stimulating the stimulus responsive material to cause the tissue portions to adhere together.

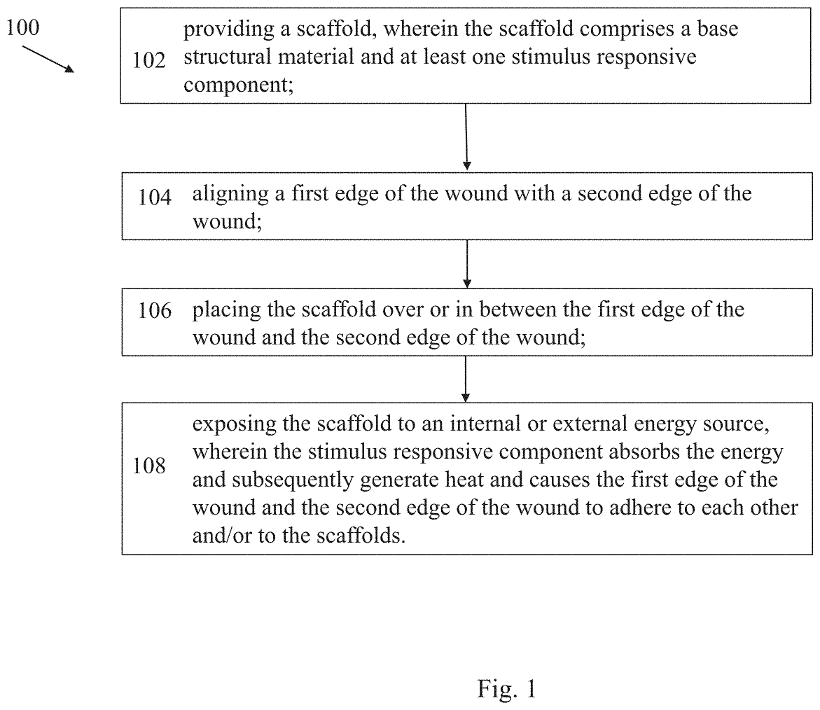

Referring now to FIG. 1, an exemplary method 100 is depicted. Method 100 begins with step 102, wherein a scaffold as described herein comprising a base structural material and at least one stimulus responsive component is provided. In step 104, a first edge of the wound is aligned with a second edge of the wound. As contemplated herein, the first edge and the second edge of the wound may be adjacent or opposing edges. In one embodiment, the first edge of the wound and the second edge of the wound may have any orientation and may be adjacent at any angle as would be understood to one skilled in the art. In step 106, the scaffold is placed over or in between the first edge of the wound and the second edge of the wound. In step 108, the stimulus responsive material is exposed to an internal or external energy source, wherein the stimulus responsive component absorbs the energy and subsequently generates heat and causes the first edge of the wound and the second edge of the wound to adhere to each other and/or to the scaffold.

In one embodiment, scaffold can be applied to the exterior of injured nerves to facilitate efficient nerve repair. In accordance with the subject invention, application of scaffold around the repair site of a cut peripheral nerve helps to direct functional axonal regeneration. In one embodiment, scaffold can be applied between severed nerve stumps. In one embodiment, scaffold can be applied both around the repair site of a cut peripheral nerve and between severed nerve stumps.

In one embodiment, scaffold can be applied for end-to-end anastomosis of nerves with small gaps (<1 cm). In one embodiment, scaffold can be applied for end-to-end anastomosis of nerves with medium gaps (1-3 cm). In one embodiment, scaffold can be applied for end-to-end anastomosis of nerves with large gaps (>3 cm).

Various external (source outside the body) and/or internal (source inside the body) stimuli that can be applied to stimulus responsive components can include: optical (e.g., light), electrical, thermal, chemical, mechanical, magnetic, acoustic, pressure, shear, biological, or enzymatic. The stimulus application can be sufficient to initiate the scaffold to weld/seal apposing wound edges of a soft tissue. In one embodiment, the opposing edges are nerve faces. These external/internal stimuli can induce crosslinking of the proteins/polypeptides/fats that are in contact with or close to the scaffold by either: (1) increasing temperature leading to a phase change in proteins/polypeptides for interdigitation; (2) initiating a chemical reaction; or (3) physically or chemically interacting with the tissue nearby in any other way. The end result achieved after exposure of the scaffold to the stimulus is a robust and a rapid tissue sealing.

In one embodiment, an internal stimulus is used to induce the tissue sealing. The scaffold includes the closure base structural material (e.g., biocompatible natural and/or semi-natural and/or synthetic polymers) with a biocompatible particle having the ability to facilitate tissue sealing upon exposure to an internal stimulus. The internal stimulus can be blood, blood components, native moisture/water, or amines and/or hydroxyls and/or carboxyl groups in the proteins, glycans, or other features of the native tissue). In one example, the scaffold can be coated, conjugated, or treated with substances or particles to expose terminal free aldehyde or epoxy groups which can interact with the amines and/or hydroxyls and/or carboxyl groups of the native proteins in the tissue allowing for tissue sealing. This can facilitate a chemical reaction which allows for tissue sealing by protein interdigitation or chemical reaction between a scaffold-tissue and tissue-tissue junction. In another example, the scaffold can be configured to expose terminal free fibrinogen or thrombin groups which can interact with the blood component in the incision site of the tissue that can cause or provide tissue welding.

The general mechanism of stimulus responsive component in response to an external or internal stimulus is to generate heat and provide heat to tissue as follows. The heat generated from an external or internal stimulus causes a physico-chemical change in the tissue (e.g., in the immediate vicinity) and interdigitiation (e.g., protein/polypeptide/fat fusion) of two ends of the tissue either with themselves or with the scaffold. The proposed mechanism is three-fold. First, at local temperatures exceeding 40° C. collagen fibrils in the tissue becomes less structured and rigid and more fluid and disorganized. Second, at local temperatures exceeding 50° C. intermolecular bonds in the tissue proteins are broken and frayed, resulting in interdigitation with the proteins/polymer of opposing tissue and the scaffold. A similar response occurs in the scaffold polymer organization, though the corresponding temperatures of these two steps may be different than for the tissue. Third, as the stimulus is removed and the local temperature decreases, these interdigitated polymers/protein bond and are strengthened, resulting in a robust tissue-tissue and/or tissue-scaffold bond. In one embodiment, the temperature can be between 40-100° C.

In one embodiment, the internal/external stimuli is optical (e.g. light). In one embodiment, the stimuli can be activated by light of wavelength 400 nm. In one embodiment, the stimuli can be activated by light of wavelength between 800 nm to 2700 nm. The wavelength of light used to activate the stimulus responsive component, may be in the visible, near-IR, mid-IR, or far-IR range.

In one embodiment, the light is a laser. A laser may deliver trains of short pulses lasting for as long as nanoseconds, microseconds or milliseconds, as appropriate. For example, illumination may be applied constantly or intermittently for a period of time in the range from about 5 seconds to about 5 mins, such as about 10 sec, about 20 sec, about 30 sec, about 40 sec, about 50 sec, about 60 sec, about 70 sec, about 80 sec, about 90 sec, about 100 sec, about 110 see, about 120 sec, about 130 sec, about 140 sec, about 150 sec, about 160 sec, about 170 sec, about 180 sec, about 200 sec, about 210 sec, about 220 sec, about 230 sec, about 240 sec, about 250 sec, about 260 sec, about 270 sec, about 280 sec, about 300 sec. In one embodiment, the interval between the laser illuminations can be between 1 second to 5 mins.

In one embodiment, the laser illumination can be applied at least once. In one embodiment, laser illumination can be applied between 1 to 10 times.

In one embodiment, laser of this invention is selected and used in a manner that maintains a desired laser radiation energy (power) at the treatment zone. In one embodiment, an average power density is between about 1 W/cm² and about 50 W/cm². Specified power density can be achieved in any suitable fashion, and preferably in a manner that provides an optimal combination of laser radiation power and beam cross-section area at the welding zone. Laser beam power at levels higher than optimal can lead to technical and economical problems. For example, if the laser radiation power exceeds several watts it may be necessary to use a cooled fiber optical cable to deliver the radiation to the welding zone, thus increasing the complexity and cost of the apparatus, as well as requiring that the user meet whatever relevant safety precautions may exist.

It is typically preferred, therefore, for reasons of efficacy, cost, and ease of use, to use a laser beam with minimal allowable cross section area. Thus, the optimal power densities at the treatment zone are reached without increasing the total power level, that results in significant reduction of safety requirements and the cost of the equipment.

The method described herein can be used for many different tissue types and areas of surgery. For example, the method can be used for, coronary arterial surgery, repair of trauma to veins and arteries, arteriovenous shunt, and intra-cranial vascular surgery. Additionally, the methods can be used for plastic surgery, surgical incision, lacerations from trauma with reduced scarring. Additionally, the method can be used to seal pulmonary air leaks and fistulas in the gastrointestinal tract, such as, but not limited to, intestinal and urinary fistulas. Furthermore, the methods described herein may also be used to seal or weld animal or human tissue including, but not limited to, skin, mucosal tissue, bone, blood vessels, neural tissue, hepatic tissue, pancreatic tissue, splenic tissue, renal tissue, bronchial tissue, tissues of the respiratory tract, tissues of the urinary tract, tissues of the gastrointestinal tract and tissues of the gynecologic tract. In one embodiment, the methods of the subject invention can be used on decellularized tissue. In one embodiment, the method described herein can be used for repair of peripheral nerve injuries and neural or nerve regeneration.

The materials and methods of the subject invention can be combined with other techniques for promoting nerve repair. These other techniques can include, for example, the application of enzymes such as chondroitin sulfate proteoglycan (CSPG) degrading enzymes and/or heparin sulfate (HSPG) degrading enzymes.

The methods described herein are suitable for use in a variety of applications, including in vitro laboratory applications, ex vivo tissue treatments, but especially in in vivo surgical procedures on living subjects, e.g., humans, animals.

The methods described herein are particularly useful for surgical applications, e.g., to seal, close, or otherwise join, two or more portions of tissue, e.g., to perform a tissue transplant and/or grafting operation, or to heal damaged tissue, e.g., a peripheral nerve injury to reattach the severed nerves. The methods described herein can be used in surgical applications where precise adhesion is necessary, and/or where the application of sutures, staples, or protein sealants is inconvenient or undesirable. For example, surgical complications such as inflammation, irritation, infection, wound gap, leakage, and epithelial ingrowth, often arise from the use of sutures.

The methods described herein are particularly suitable for use in surgery or microsurgery, for example, in surgical operations or maneuvers of the severed nerves.

As another example, sutures cannot be satisfactorily used on bone joint cartilage because of their mechanical interference with the mutual sliding of cartilage surfaces required for joint motion. Neither can sutures be used to seal surfaces of small blood vessels with diameters 1-2 mm or less, as sutures impinge upon the vessel lumen, compromising blood flow. Further, in skin grafting, sutures can induce foreign body responses that lead to scarring and therefore reduce cosmetic value. Thus, the methods described herein are also useful in surgical interventions of vascular tissue, joint cartilage, skin, gastrointestinal tract, nerve sheaths, urological tissue, small ducts (urethra, ureter, bile ducts, thoracic duct), oral tissue or even tissues of the middle or inner ear. Other procedures where sutures or staples are not indicated or desirable, and where the methods described herein are useful, include procedures involving laparoscopic operations or interventions such as laparoscopic (LP) thoracic procedures, LP appendectomy, LP hernia repairs, LP tubal ligations and LP orbital surgeries.

The methods of the present invention as described herein are optimal for the repair of musculoskeletal tissues such as tendons, ligaments, extracellular matrix and cartilage. For example, these methods are particularly suitable for repair of lacerations or ruptures of tendons such that the healing of the tendon in the patient may benefit from an immediate recovery in the strength of the injured site following repair, and such that the recovery is not hindered by infection of foreign-body reactions that may occur following the use of multiple staples or sutures. In addition, use of these methods may reduce the surgery time, may help prevent a future recurrent rupture of the site, and may reduce hospitalization and immobilization time during the rehabilitation period. In one embodiment, the methods as described herein are optimal for use in sports medicine.

The methods described herein can also be used in tissue grafting. Exogenous grafts can be, for example, autografts, allografts or xenografts. In one embodiment, an exogenous tissue graft comprising tissue such as skin, muscle, vasculature, stomach, esophagus, colon or intestine, can be placed over the surface of the wound, covered with the scaffold as described herein comprising a base structure and at least one stimulus responsive component, and activated with a stimulation such as visible light source, e.g., an incandescent, fluorescent or mercury vapor light source, e.g., a xenon arc lamp, or a laser light source, e.g. argon-ion laser. In one embodiment, the method of present invention enables rapid and sustained adherence of the graft to the tissue surface and the ability to resist shear stress. Sources of grafted tissue can be any known in the art, including exogenous grafts obtained from non-injured tissues in a subject. Sources of grafted tissue can also comprise extracellular matrix-based scaffolds, such as collagen and proteoglycan, and/or other engineered tissue implants.

Exogenous grafts can likewise be synthetic. Synthetic materials suitable for use in grafting include, but are not limited to, silicon, polyurethane, polyvinyl and nylon.

The methods described herein can also be used to supplement the use of sutures, e.g., to reinforce sutured anastomosis. Sutures leave a tract behind which can allow for leakage of fluids and organisms. The problem of leakage is especially critical in vascular anastomoses or for any anastomoses of a fluid-containing structure (aorta, ureter, GI tract, eye, etc.) where the fluid or contents inside can leak out through the suture hole. In one embodiment, a wound can be sutured according to general procedures and then treated with the methods described herein, thereby making the healing wound water tight, and impermeable to bacteria.

In addition, the methods described herein can be used in non-surgical wound healing applications, e.g., wound healing in addition to, or in place of, a conventional bandage, optionally in combination with another beneficial material for wound healing. In one embodiment, scaffold can be applied to a wound, and activated with a stimulator such as visible light source, e.g., an incandescent, fluorescent or mercury vapor light source, e.g., a xenon arc lamp, or a laser light source. The bandage can contain another beneficial material for wound healing, e.g., an antibiotic. In some embodiments, the bandage, and/or the light source, can be supplied to a subject in a kit, e.g., a kit for use by a health care practitioner, or a kit for household use, which kits can contain instructions for use. The scaffold described herein can be left on the wound, or can be replaced as necessary. Such an adhesive can be used ex vivo, on a tissue removed from the body, or in situ on a subject, e.g., a human subject. For example, a scaffold described herein can be used as an “artificial skin” or covering agent to cover large, oozing surfaces inside or outside the body.

The methods described herein can also be used to cross-link proteins for use in laboratory applications, e.g., to fix proteins for microscopy; to immobilize antibodies or other protein reagents to a substrate for diagnosis or purification; or to cross link proteins or peptides to a solid matrix for use in chromatographic or immunological applications.

### Composition

The present invention provides compositions and methods for promoting the repair and growth of nerve tissue using a scaffold, wherein the scaffold comprises a base structural material and at least one stimulus responsive component. In one aspect, the invention includes a scaffold, including but not limited to for example a polymeric matrix, a hydrogel, a film, an electrospun scaffold, a tubular conduit and the like, wherein the scaffold is used to align the edges of the tissues, restore the continuity of tissues interrupted by disease, traumatic events or surgical procedures and act as a bioadhesive, able to hold both ends of a cut nerve adjacent to each other until the nerve is fully healed. In one embodiment, the tissue is a nerve tissue.

In one aspect the present invention relates to a composition that can be responsive to a stimulus, such as photothermal excitation (e.g., laser/light excitation), to enhance the ability of a scaffold to hold the two nerve ends together and improve healing. In one embodiment, the scaffold is applied around the repair site of a cut peripheral nerve helps to direct functional axonal regeneration. In one embodiment, the scaffold can be applied between severed nerve stumps. In one embodiment, scaffold can be applied both around the repair site of a cut peripheral nerve and between severed nerve stumps. In one embodiment, stimulus responsive component is a nanoparticle.

In one exemplary embodiment, the stimulus responsive nanoparticles can be responsive to photothermal excitation in order to provide an additional benefit of enhancing nerve repair via photothermal tissue welding; however, the nanoparticles may have different compositions that are simulated by different stimuli as described herein.

In one embodiment, scaffold according to the present invention may be in the form of a film or a gel. The present invention also encompasses compositions in the form of a “pre-gel”, which may be dehydrated to form a gel or a film. Films or gels according to the present invention may be useful for repairing or strengthening nerve tissue or for joining discontinuous portions of nerve tissue.

In one embodiment, scaffold according to the present invention may be in form of an electrospun scaffold. In one embodiment, scaffold according to the present invention are composed of aligned fibers.

In one embodiment of the present invention, the scaffold can be formed from a biocompatible polymer. A variety of biocompatible polymers can be used to make the scaffold according to the present invention including synthetic polymers, natural polymers or combinations thereof.

Examples of synthetic polymers include polyanhydrides, polyhydroxyacids such as polylactic acid, polyglycolic acids and copolymers thereof, polyesters, polyamides, polyorthoesters, and some polyphosphazenes. Examples of naturally occurring polymers include proteins and polysaccharides such as collagen, hyaluronic acid, albumin and gelatin. The one or more agents can be encapsulated within, throughout, and/or on the surface of the polymers.

In some embodiments, the synthetic polymer or copolymer is prepared from at least one of the group of monomers consisting of acrylic acid, methacrylic acid, ethyleneimine, crotonic acid, acrylamide, ethyl acrylate, methyl methacrylate, 2-hydroxyethyl methacrylate, lactic acid, glycolic acid, .ε-caprolactone, acrolein, cyanoacrylate, bisphenol A, epichlorhydrin, hydroxyalkylacrylates, siloxane, dimethylsiloxane, ethylene oxide, ethylene glycol, hydroxyalkyl-methacrylates, N-substituted acrylamides, N-substituted methacrylamides, N-vinyl-2-pyrrolidone, 2,4-pentadiene-1-ol, vinyl acetate, acrylonitrile, styrene, p-amino-styrene, p-amino-benzyl-styrene, sodium styrene sulfonate, sodium 2-sulfoxyethyl methacrylate, vinyl pyridine, aminoethyl methacrylates, 2-methacryloyloxy-trimethylammonium chloride, N,N′-methylenebisacrylamide-, ethylene glycol dimethacrylates, 2,2′-(p-phenylenedioxy)-diethyl dimethacrylate, divinylbenzene, and triallylamine, methylenebis-(4-phenyl-isocyanate).

A variety of polymers from synthetic and/or natural sources can be used to produce the scaffold of the invention. For example, lactic or polylactic acid or glycolic or polyglycolic acid can be utilized to form poly(lactide) (PLA) or poly(L-lactide) (PLLA) nanofibers or poly(glycolide) (PGA) nanofibers. The polymer matrix can also be made from more than one monomer or subunit thus forming a co-polymer, terpolymer, etc. For example, lactic or polylactic acid and be combined with glycolic acid or polyglycolic acid to form the copolymer poly(lactide-co-glycolide) (PLGA). Other copolymers of use in the invention include poly(ethylene-co-vinyl) alcohol). In an exemplary embodiment, the scaffold can comprise a polymer or subunit which is a member selected from an aliphatic polyester, a polyalkylene oxide, polydimethylsiloxane, polyvinylalcohol, polylysine, and combinations thereof. In another exemplary embodiment, the scaffold can comprise two different polymers or subunits which are members selected from an aliphatic polyester, a polyalkylene oxide, polydimethylsiloxane, polyvinylalcohol, polylysine, and combinations thereof. In another exemplary embodiment, the scaffold comprises three different polymers or subunits which are members selected from an aliphatic polyester, a polyalkylene oxide, polydimethylsiloxane, polyvinylalcohol, polylysine, and combinations thereof. In an exemplary embodiment, the aliphatic polyester is linear or branched. In another exemplary embodiment, the linear aliphatic polyester is a member selected from lactic acid (D- or L-), lactide, poly(lactic acid), poly(lactide) glycolic acid, poly(glycolic acid), poly(glycolide), glycolide, poly(lactide-co-glycolide), poly(lactic acid-co-glycolic acid), polycaprolactone and combinations thereof. In another exemplary embodiment, the aliphatic polyester is branched and comprises at least one member selected from lactic acid (D- or L-), lactide, poly(lactic acid), poly(lactide) glycolic acid, poly(glycolic acid), poly(glycolide), glycolide, poly(lactide-co-glycolide), poly(lactic acid-co-glycolic acid), polycaprolactone and combinations thereof which is conjugated to a linker or a biomolecule.

As another example, the polymer may be formed from functionalized polyester graft copolymers. The functionalized graft copolymers are copolymers of polyesters, such as poly(glycolic acid) or poly(lactic acid), and another polymer including functionalizable or ionizable groups, such as a poly(amino acid). In another embodiment, polyesters may be polymers of α-hydroxy acids such as lactic acid, glycolic acid, hydroxybutyric acid and valeric acid, or derivatives or combinations thereof. The inclusion of ionizable side chains, such as polylysine, in the polymer has been found to enable the formation of more highly porous particles, using techniques for making microparticles known in the art, such as solvent evaporation. Other ionizable groups, such as amino or carboxyl groups, may be incorporated, covalently or noncovalently, into the polymer to enhance porosity. For example, polyaniline could be incorporated into the polymer. These groups can be modified further to contain hydrophobic groups capable of binding load molecules.

In some embodiments, the polymer can include one or more of the following: polyphosphazines, poly(vinyl alcohols), polyamides, polycarbonates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes, poly-ε-caprolactone, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly(ethylene terephthalate), poly(vinyl acetate), polyvinyl chloride, polystyrene, polyvinyl pyrrolidone, pluronics, polyvinylphenol, saccharides (e.g., dextran, amylose, hyalouronic acid, poly(sialic acid), heparans, heparins, etc.); poly (amino acids), e.g., poly(aspartic acid) and poly(glutamic acid); nucleic acids and copolymers thereof.

In some embodiments, the polymer can include one or more of the following: peptide, saccharide, poly(ether), poly(amine), poly(carboxylic acid), poly(alkylene glycol), such as poly(ethylene glycol) (“PEG”), poly(propylene glycol) (“PPG”), copolymers of ethylene glycol and propylene glycol and the like, poly(oxyethylated polyol), poly(olefinic alcohol), poly(vinylpyrrolidone), poly(hydroxypropylmethacrylamide), poly(a-hydroxy acid), poly(vinyl alcohol), polyphosphazene, polyoxazoline, poly(N-acryloylmorpholine), polysialic acid, polyglutamate, polyaspartate, polylysine, polyethyeleneimine, biodegradable polymers (e.g., polylactide, polyglyceride and copolymers thereof), polyacrylic acid.

In various embodiments, the scaffolds can be modified with one or more functional groups for covalently attaching a variety of proteins (e.g., collagen) or compounds such as therapeutic agents. Therapeutic agents which may be linked to the scaffold include, but are not limited to, analgesics, anesthetics, antifungals, antibiotics, anti-inflammatories, anthelmintics, antidotes, antiemetics, antihistamines, anti-cancer drugs, antihypertensives, antimalarials, antimicrobials, antipsychotics, antipyretics, antiseptics, antiarthritics, antituberculotics, antitussives, antivirals, cardioactive drugs, cathartics, chemotherapeutic agents, a colored or fluorescent imaging agent, corticoids (such as steroids), antidepressants, depressants, diagnostic aids, diuretics, enzymes, expectorants, hormones, hypnotics, minerals, nutritional supplements, parasympathomimetics, potassium supplements, radiation sensitizers, a radioisotope, fluorescent nanoparticles, sedatives, sulfonamides, stimulants, sympathomimetics, tranquilizers, urinary anti-infectives, vasoconstrictors, vasodilators, vitamins, xanthine derivatives, and the like. The therapeutic agent may also be other small organic molecules, naturally isolated entities or their analogs, organometallic agents, chelated metals or metal salts, peptide-based drugs, or peptidic or non-peptidic receptor targeting or binding agents. It is contemplated that linkage of the therapeutic agent to the scaffold may be via a protease sensitive linker or other biodegradable linkage. Molecules which may be incorporated into the biomimetic scaffold include, but are not limited to, vitamins and other nutritional supplements; glycoproteins (e.g., collagen); fibronectin; peptides and proteins; carbohydrates (both simple and/or complex); proteoglycans; antigens; oligonucleotides (sense and/or antisense DNA and/or RNA); antibodies (for example, to infectious agents, tumors, drugs or hormones); and gene therapy reagents.

In various embodiments, the scaffolds can further comprise one or more polysaccharides, including glycosaminoglycans (GAGs) or glycosaminoglycans, with suitable viscosity, molecular mass, and other desirable properties. The term “glycosaminoglycan” is intended to encompass any glycan (i.e., polysaccharide) comprising an unbranched polysaccharide chain with a repeating disaccharide unit, one of which is always an amino sugar. These compounds as a class carry a high negative charge, are strongly hydrophilic, and are commonly called mucopolysaccharides. This group of polysaccharides includes heparin, heparan sulfate, chondroitin sulfate, dermatan sulfate, keratan sulfate, and hyaluronic acid. These GAGs are predominantly found on cell surfaces and in the extracellular matrix. The term “glucosaminoglycan” is also intended to encompass any glycan (i.e. polysaccharide) containing predominantly monosaccharide derivatives in which an alcoholic hydroxyl group has been replaced by an amino group or other functional group such as sulfate or phosphate. An example of a glucosaminoglycan is poly-N-acetyl glucosaminoglycan, commonly referred to as chitosan. Exemplary polysaccharides that may be useful in the present invention include dextran, heparan, heparin, hyaluronic acid, alginate, agarose, carageenan, amylopectin, amylose, glycogen, starch, cellulose, chitin, chitosan and various sulfated polysaccharides such as heparan sulfate, chondroitin sulfate, dextran sulfate, dermatan sulfate, or keratan sulfate.

In one embodiment, scaffold is in form of a film. In one embodiment, the film comprises chitosan. In one embodiment, the chitosan film may comprise 1.0 wt % aqueous acetic acid. In one embodiment, chitosan film comprises 2.0 wt % chitosan. In one embodiment, chitosan film comprises 1 w/v % glutaraldehyde. In one embodiment, glutaraldehyde is diluted in 0.05%. In one embodiment, chitosan film comprises 1 w/w % nanoparticles. In one embodiment, nanoparticle is gold nanoparticle. In one embodiment, nanoparticle is silver nanoparticle.

In one embodiment, scaffold is in the form of a film. In one embodiment, the film comprises silk. In one embodiment, silk film comprises 2.0 wt % silk dissolved in nano-pure water. In one embodiment, silk film comprises 1 w/v % glutaraldehyde. In one embodiment, glutaraldehyde is diluted in 0.05%. In one embodiment, silk film comprises 1 w/w % nanoparticles. In one embodiment, nanoparticle is gold nanoparticle. In one embodiment, nanoparticle is silver nanoparticle.

In one embodiment, scaffold is in form of a film. In one embodiment, the film comprises cellulose. In one embodiment, cellulose film comprises 2.0 wt % cellulose dissolved in nano-pure water. In one embodiment, cellulose film comprises 1 w/v % glutaraldehyde. In one embodiment, glutaraldehyde is diluted in 0.05%. In one embodiment, cellulose film comprises 1 w/w % nanoparticles. In one embodiment, nanoparticle is gold nanoparticle. In one embodiment, nanoparticle is silver nanoparticle.

In one embodiment, scaffold is in form of a film. In one embodiment, the film comprises alginate. In one embodiment, alginate film comprises 2.0 wt % alginate dissolved in nano-pure water. In one embodiment, alginate film comprises 1 w/v % glutaraldehyde. In one embodiment, glutaraldehyde is diluted in 0.05%. In one embodiment, alginate film comprises 1 w/w % nanoparticles. In one embodiment, nanoparticle is gold nanoparticle. In one embodiment, nanoparticle is silver nanoparticle.

In various embodiments, the scaffold may be porous. Porosity may be accomplished by a variety of methods. Non-limiting examples of such processes include solvent casting/salt leaching, electrodeposition, and thermally induced phase separation.

In various embodiments, the scaffolds can further comprise one or more extracellular matrix materials and/or blends of naturally occurring extracellular matrix materials, including but not limited to collagen, fibrin, fibrinogen, thrombin, elastin, laminin, fibronectin, hyaluronic acid, chondroitin 4-sulfate, chondroitin 6-sulfate, dermatan sulfate, heparin sulfate, heparin, and keratan sulfate, proteoglycans, and combinations thereof. Some collagens that may be beneficial include but are not limited to collagen types I, II, III, IV, V, VI, VII, VIII, IX, X, XI, XII, XIII, XIV, XV, XVI, XVII, XVIII, and XIX. These proteins may be in any form, including but not limited to native and denatured forms. The scaffolds can further comprise one or more carbohydrates such as chitin, chitosan, alginic acids, and alginates such as calcium alginate and sodium alginate. These materials may be isolated from plant products, humans or other organisms or cells or synthetically manufactured. Also contemplated are crude extracts of tissue, extracellular matrix material, or extracts of non-natural tissue, alone or in combination. Extracts of biological materials, including but are not limited to cells, tissues, organs, and tumors may also be included.

In some embodiments, the scaffolds can further comprise one or more natural or synthetic drugs, such as nonsteroidal anti-inflammatory drugs (NSAIDs). In one embodiment, the scaffolds can further comprise antibiotics, such as penicillin.

In one embodiment, the scaffolds can further comprise natural peptides, such as glycyl-arginyl-glycyl-aspartyl-serine (GRGDS), arginylglycylaspartic acid (RGD), and amelogenin. In one embodiment, the scaffolds can further comprise proteins, such as chitosan and silk. In one embodiment, the scaffolds can further comprise sucrose, fructose, cellulose, or mannitol. In one embodiment, the scaffolds can further comprise extracellular matrix proteins, such as fibronectin, vitronectin, laminin, collagens, and vixapatin (VP12). In one embodiment, the scaffolds can further comprise disintegrins, such as VLO4. In one embodiment, the scaffolds can further comprise decellularized or demineralized tissue. In one embodiment, the scaffolds can further comprise synthetic peptides, such as emdogain.

In one embodiment, the scaffolds can further comprise nutrients, such as bovine serum albumin. In one embodiment, the scaffolds can further comprise vitamins, such as vitamin B2, vitamin Ad, Vitamin D, Vitamin E, and Vitamin K. In one embodiment, the scaffold can further comprise nucleic acids, such as mRNA and DNA. In one embodiment, the scaffolds can further comprise natural or synthetic steroids and hormones, such as dexamethasone, hydrocortisone, estrogens, and its derivatives. In one embodiment, the scaffold can further comprise growth factors, such as fibroblast growth factor (FGF), transforming growth factor beta (TGF-β). In one embodiment, the scaffolds can further comprise a delivery vehicle, such as nanoparticles, microparticles, liposomes, viral and non-viral transfection systems.

In one embodiment, scaffold can be biodegradable and/or bioabsorbable. This can allow for the scaffold to be degraded and possibly excreted from the body after prolonged exposure to bodily fluids, cells, or other substances.

### Hydrogels

In one embodiment, the present invention provides a scaffold in form of a hydrogel comprising at least one stimulus responsive component.

Hydrogels can generally absorb much fluid and, at equilibrium, typically are composed of 60-90% fluid and only 10-30% polymer. In one embodiment, the water content of hydrogel is about 70-80%. Hydrogels are particularly useful due to the inherent biocompatibility of the polymeric network. Hydrogel biocompatibility can be attributed to hydrophilicity and ability to imbibe large amounts of biological fluids. In certain embodiments, the hydrogels can be prepared by crosslinking hydrophilic biopolymers or synthetic polymers. In certain embodiments, construction of hydrogels comprises the polymerization and/or copolymerization of monomers, macromers, polymers and the like. For example, in one embodiment hydrogel formation comprises copolymerization of two or more types of biopolymers and/or synthetic polymers.

Hydrogels may be prepared by crosslinking hydrophilic biopolymers or synthetic polymers. Examples of the hydrogels formed from physical or chemical crosslinking of hydrophilic biopolymers include but are not limited to, hyaluronans, chitosans, alginates, collagen, dextran, pectin, carrageenan, polylysine, gelatins, fibrin, or agarose. Examples of hydrogels based on chemical or physical crosslinking synthetic polymers include but are not limited to (meth)acrylate-oligolactide-PEO-oligolactide-(meth)acrylate, poly(ethylene glycol) (PEO), poly(propylene glycol) (PPO), PEO-PPO-PEO copolymers (Pluronics), poly(phosphazene), poly(methacrylates), poly(N-vinylpyrrolidone), PL(G)A-PEO-PL(G)A copolymers, or poly(ethylene imine).

In one embodiment, the hydrogel comprises at least one biopolymer. In other embodiments, the hydrogel comprises at least two biopolymers. In yet other embodiments, the hydrogel comprises at least one biopolymer and at least one synthetic polymer. In one embodiment, the hydrogel comprises at least two synthetic polymers.

Hydrogels closely resemble the natural living extracellular matrix. Hydrogels can also be made degradable in vivo by incorporating PLA, PLGA or PGA polymers. Moreover, hydrogels can be modified with fibronectin, laminin, vitronectin, or, for example, RGD for surface modification, which can promote cell adhesion and proliferation. Indeed, altering molecular weights, block structures, degradable linkages, and cross-linking modes can influence strength, elasticity, and degradation properties of the instant hydrogels.

In certain embodiments, one or more multifunctional cross-linking agents may be utilized as reactive moieties that covalently link biopolymers or synthetic polymers. Such bifunctional cross-linking agents may include glutaraldehyde, epoxides (e.g., bis-oxiranes), oxidized dextran, p-azidobenzoyl hydrazide, N-[α.-maleimidoacetoxy]succinimide ester, p-azidophenyl glyoxal monohydrate, bis-[β-(4-azidosalicylamido)ethyl]disulfide, bis[sulfosuccinimidyl]suberate, dithiobis[succinimidyl proprionate, disuccinimidyl suberate, 1-ethyl-3-[3-dimethylaminopropyl]carbodiimide hydrochloride (EDC), N-hydroxysuccinimide (NETS) and other bifunctional cross-linking reagents known to those skilled in the art. It should be appreciated by those in skilled in the art that the mechanical properties of the hydrogel are greatly influenced by the cross-linking time and the amount of cross-linking agents.

In another embodiment utilizing a cross-linking agent, polyacrylated materials, such as ethoxylated (20) trimethylpropane triacrylate, may be used as a non-specific photo-activated cross-linking agent. Components of an exemplary reaction mixture would include a thermoreversible hydrogel held at 39° C., polyacrylate monomers, such as ethoxylated (20) trimethylpropane triacrylate, a photo-initiator, such as eosin Y, catalytic agents, such as 1-vinyl-2-pyrrolidinone, and triethanolamine. Continuous exposure of this reactive mixture to long-wavelength light (>498 nm) would produce a cross-linked hydrogel network.

In one embodiment, the hydrogel comprises a UV sensitive curing agent which initiates hydrogel polymerization. For example, in one embodiment, a hydrogel comprises the photoinitiator 4-(2-hydroxyethoxy)phenyl-(2-hydroxy-2-propyl)ketone. In one embodiment, polymerization is induced by 4-(2-hydroxyethoxy)phenyl-(2-hydroxy-2-propyl)ketone upon application of UV light. Other examples of UV sensitive curing agents include 2-hydroxy-2-methyl-1-phenylpropan-2-one, 4-(2-hydroxyethoxy)phenyl (2-hydroxy-2-phenyl-2-hydroxy-2-propyl)ketone, 2,2-dimethoxy-2-phenyl-acetophenone 1-[4-(2-Hydroxyethoxy)-phenyl]-2-hydroxy-2-methyl-1-propane-1-one, 1-hydroxycyclohexylphenyl ketone, trimethyl benzoyl diphenyl phosphine oxide and mixtures thereof.

In one embodiment, the hydrogel may be further stabilized and enhanced through the addition of one or more enhancing agents. The term “enhancing agent” or “stabilizing agent” refers to any compound added to the hydrogel scaffold, in addition to the high molecular weight components, that enhances the hydrogel scaffold by providing further stability or functional advantages. The enhancing agent may include any compound, such as polar compounds, that enhance the hydrogel by providing further stability or functional advantages when incorporated in the cross-linked hydrogel.

Preferred enhancing agents for use with hydrogel include polar amino acids, amino acid analogues, amino acid derivatives, intact collagen, and divalent cation chelators, such as ethylenediaminetetraacetic acid (EDTA) or salts thereof. Polar amino acids include tyrosine, cysteine, serine, threonine, asparagine, glutamine, aspartic acid, glutamic acid, arginine, lysine, or histidine. In one embodiment, the contemplated polar amino acids are L-cysteine, L-glutamic acid, L-lysine, and L-arginine. Polar amino acids, EDTA, and mixtures thereof, are also contemplated enhancing agents. The enhancing agents may be added to the hydrogel before or during the crosslinking of the high molecular weight components. The hydrogel may exhibit an intrinsic bioactivity, which may be a function of the unique stereochemistry of the cross-linked macromolecules in the presence of the enhancing and strengthening polar amino acids, as well as other enhancing agents.

In certain embodiments, the hydrogel is modified to improve its functionality. For example, the hydrogel may be coated with any number of compounds in order enhance its biocompatibility, reduce its immunogenicity, enhance stability, enhance degradation, and/or enhance drug delivery.

In one embodiment the stimulus responsive component may be added to the hydrogel solution prior to gelation or polymerization of the gel. In one embodiment, the stimulus responsive component may be added to hydrogel solution in any amount desired to produce a desired effect.

### Fibers

In one embodiment, the present invention provides a scaffold in form of fibers comprising at least one stimulus responsive component.

Fibers may be produced using any method known in the art such as, melt spinning, extrusion, drawing, wet spinning or electrospinning. Alternatively, as the concentrated solution has a gel-like consistency, a fiber can be pulled directly from the solution. In one embodiment, the fibers are produced using electrospinning.

Electrospinning can be performed by any means known in the art. In one embodiment, a steel capillary tube with a 1.0 mm internal diameter tip is mounted on an adjustable, electrically insulated stand. In one embodiment, the capillary tube is maintained at a high electric potential and mounted in the parallel plate geometry. In one embodiment, the capillary tube is connected to a syringe filled with fibrous scaffold material solution. A constant volume flow rate is maintained using a syringe pump, set to keep the solution at the tip of the tube without dripping. The electric potential, solution flow rate, and the distance between the capillary tip and the collection screen are adjusted so that a stable jet is obtained. Dry or wet fibers are collected by varying the distance between the capillary tip and the collection screen.

A collection screen suitable for collecting fibrous scaffold material fibers can be a wire mesh, a polymeric mesh, or a water bath. Alternatively, the collection screen is an aluminum foil. The aluminum foil can be coated with Teflon fluid to make peeling off the fibrous scaffold material fibers easier. One skilled in the art will be able to readily select other means of collecting the fiber solution as it travels through the electric field. The electric potential difference between the capillary tip and the aluminum foil counter electrode is, preferably, gradually increased to about 12 kV, however, one skilled in the art can adjust the electric potential to achieve suitable jet stream.

Electrospinning for the formation of fine fibers has been actively explored recently for applications such as high-performance filters and biomaterial scaffolds for cell growth, vascular grafts, wound dressings or tissue engineering. Fibers with a nanoscale diameter provide benefits due to their high surface area. In this electrostatic technique, a strong electric field is generated between a polymer solution contained in a syringe with a capillary tip and a metallic collection screen. When the voltage reaches a critical value, the charge overcomes the surface tension of the deformed drop of suspended polymer solution formed on the tip of the syringe, and a jet is produced. The electrically charged jet undergoes a series of electrically induced bending instabilities during passage to the collection screen that results in stretching. This stretching process is accompanied by the rapid evaporation of the solvent and results in a reduction in the diameter of the jet. The dry fibers accumulated on the surface of the collection screen form a non-woven mesh of nanometer to micrometer diameter fibers even when operating with aqueous solutions at ambient temperature and pressure. The electrospinning process can be adjusted to control fiber diameter by varying the charge density and polymer solution concentration, while the duration of electrospinning controls the thickness of the deposited mesh.

Protein fiber spinning in nature, such as for silkworm and spider silks, is based on the formation of concentrated solutions of metastable lyotropic phases that are then forced through small spinnerets into air. The fiber diameters produced in these natural spinning processes range from tens of microns in the case of silkworm silk to microns to submicron in the case of spider silks. The production of fibers from protein solutions has typically relied upon the use of wet or dry spinning processes. Electrospinning offers an alternative approach to protein fiber formation that can potentially generate very fine fibers. This can be a useful feature based on the potential role of these types of fibers in some applications such as biomaterials and tissue engineering.

Fibers or fiber bundles can be braided, twisted, or manipulated by one of skill in the art to be grouped together or stand individually for the formation of scaffolds. One of skill in the art can form scaffolds using any configuration of fibers that is desired (e.g., aligned fibers, braided, twisted, random etc.).

### Stimulus Responsive Component

The present invention comprises use of stimulus responsive material to enhance the repair and growth of nerve tissue using a scaffold, wherein the scaffold comprises a base structural material and at least one stimulus responsive component.

The scaffolds can be used for surgical approximation, repair and regeneration of nerve tissue by achieving stimuli responsive tissue-integrating closure that can provide for rapid sealing of soft tissue.

In one aspect, the stimulus responsive material is coated, embedded, crosslinked, or otherwise associated with the structural material. In one aspect, the stimulus responsive material is in a particle form. In one embodiment, the stimulus responsive particle is a nanoparticle (e.g., nanosphere, nanorod, etc.).

In one embodiment, the nanoparticles may be responsive to two or more stimuli, such that one or more stimuli can be applied to obtain the response from the nanoparticle. In one aspect, two or more different nanoparticles may be included in the structural material that are responsive to two or more different stimuli. As such, a first nanoparticle may be responsive to a first stimuli, and a second nanoparticle may be responsive to a different second stimuli. This can allow for using one or two different stimuli to induce sealing and healing responses. The different stimuli can be used at the same time, different times, in sequence, or in patterns to promote enhanced nerve repair.

Examples of materials that can be responsive to an optical (e.g., light) stimulus can include: gold nanorods, gold nanoparticles, gold nanospheres, indocyanin green, neodymium-doped nanoparticles (Nd:NPs), carbon nanotubes (CNTs), organic nanoparticles (O:NPs), gold nanostars (GNSs), alumina nanoparticles, copper nanoparticles, silver nanoplates/prisms, silver nanoparticles or near-infrared absorbing dyes (absorbance of the dye between 650-1350 nm). Many materials have a range of wavelengths to which they are responsive, and may be tuned to a specific wavelength.

In one embodiment, laser light energy is converted to heat (e.g., photothermal conversion). Photoresponsive scaffolds are generated by adding and/or reinforcing and/or doping and/or coating the base structural material (e.g., biocompatible natural and/or semi-natural and/or synthetic polymers) with light absorbing elements. The light stimulus response materials can advantageously absorb in the optical window of 600-1200 nm light wavelength and convert the laser energy into heat. The heat produced causes a physico-chemical change in the tissue (e.g., in the immediate vicinity of the scaffold) leading to interdigitiation (e.g., protein/polypeptide/fat fusion) of two ends of the tissue that results in tissue welding. In one embodiment, a continuous laser wave can be used. In one embodiment a pulsed laser wave can be used.

The nanoparticles of the present invention have dimensions of between 1-5000 nm. The excitation light used in typically NIR, although other excitation may be used such as the rest of the IR spectrum, UV, and VIS or combinations thereof. Typically, the light is in the wavelength range of 600-2000 nm. The particles are ideally of nanometer-scale dimensions. Well-known examples are colloids such as gold colloids and silver colloids. Alternatively, the nanoparticles may be nanoshells. The method typically involves the use of nanoparticles of one composition; however, nanoparticles of more than one composition may be used. If more than one composition of nanoparticles is used, it is typical for the different compositions to all absorb at least one common wavelength; however, this is not absolutely necessary. As a result, the temporal heating profiles of the different nanoparticles may be the same or different. Typically, the temporal heating profiles are the same.

Ideally, to maximize penetration of light through the depth of the tissue and to minimize damage to surrounding tissue, one would prefer to use a laser light source that is not appreciably absorbed by tissues. This can be accomplished using NIR light, specifically in the wavelength region between 600-2000 nm, where penetration of light into tissue is maximal. Exposure to light at these wavelengths will not generate significant heating in tissues, and thus will not induce tissue damage. However, when light at these wavelengths interacts with nanoparticles designed to strongly absorb NIR light, heat will be generated rapidly and sufficiently to induce tissue welding. Because NIR wavelengths of light are highly transmitted through tissue, it is possible to access and treat tissue surfaces that are otherwise difficult or impossible.

Several new classes of such nanoparticles that offer more specific and accurate repair technologies, based on nanoparticles that emit or scatter NIR light and that can be easily conjugated to antibodies, as well as highly localized, targeted, and minimally invasive treatment strategies based on photothermal interactions with nanoparticles, have been developed. In one embodiment, the nanoparticles are nanoshells and are formed with a core of a dielectric or inert material such as silicon, coated with a material such as a highly conductive metal which can be excited using radiation such as NIR light (approximately 700 to 1500 nm). Other nanoparticles absorb across other regions of the electromagnetic spectrum such as the ultraviolet or visible region. Upon excitation, the nanoshells emit heat. The combined diameter of the shell and core of the nanoshells typically ranges from the tens to the hundreds of nanometers. The nanoparticles have dimension of from 1 to 5000 nanometers.

NIR light is advantageous for its ability to penetrate tissue. Other types of radiation can also be used, depending on the selection of the nanoparticle coating and targeted cells. Examples include x-rays, magnetic fields, electric fields, and ultrasound. The problems with the existing methods for hyperthermia, such as the use of heated probes, microwaves, ultrasound, lasers, perfusion, radiofrequency energy, and radiant heating is avoided since the levels of radiation used as described herein is insufficient to induce hyperthermia except at the surface of the nanoparticles, where the energy is more effectively concentrated by the metal surface on the dielectric. The currently available methods suffer from the use of generalized as opposed to localized heating or the need for high power radiation sources or both. Targeting molecules can be antibodies or fragments thereof, ligands for specific receptors, or other proteins specifically binding to the surface of the cells to be targeted.

In one embodiment, magnetic energy is converted to heat (e.g., magnetothermal). A scaffold can include particles that convert magnetic energy to heat by adding and/or reinforcing and/or doping and/or coating the base structural material (e.g., biocompatible natural and/or semi-natural and/or synthetic polymers) with a biocompatible particle. The particle can be organic dyes, inorganic dyes, or organic nanoparticles or inorganic nanoparticles, or ferromagnetic particles or anti-ferromagnetic particles (e.g., 1-100 nm longest dimension) that absorb the incident magnetic field to produce heat and/or initiate a chemical reaction which allows for tissue welding and sealing by protein interdigitation or chemical reaction between a scaffold-tissue and tissue-tissue junction.

In one embodiment, electrical energy is converted to heat (e.g., electrothermal). A scaffold can include particles with resistive elements that can convert electrical energy into heat or and/or initiate a chemical reaction which allows for tissue welding and sealing by protein interdigitation or chemical reaction between a scaffold-tissue and tissue-tissue junction. The particles having the resistive elements can be included in the scaffold by adding and/or reinforcing and/or doping and/or coating the base structural material (e.g., biocompatible natural and/or semi-natural and/or synthetic polymers) with a biocompatible particle having the resistive element. The resistive element can be any organic dyes, inorganic dyes, or organic nanoparticles or inorganic nanoparticles, or ferromagnetic particles or anti-ferromagnetic particles (e.g., 1-100 nm longest dimension) that absorb the electrical energy and convert the electrical energy as described herein.

In one embodiment, the stimulus responsive component can include the nanoparticles at various sizes, concentrations, amounts, distributions, or arrangements in the structural material. The modulation of the nanoparticles in size, amount, or type can be used to control the response to the stimulus. In one example, when the nanoparticle generates heat in response to the stimulus, the control of the nanoparticles can be used to control the heat generation from the stimulus. As such, the control of the nanoparticles can provide accurate control of heat generation or stimuli responsiveness. Also, the methods of use can include modulating the power or intensity or time of application of the stimulus to modulate the heat generation. The modulation of the stimulus can be conducted during the surgical procedure, where the temperature of the wound and/or closure device can be monitored with a temperature monitoring device, and the application of the stimulus can be modulated in order to modulate the temperature. Modulation of the stimulus may be conducted along with modulation of the inclusion of the nanoparticles in the structural material.

In one embodiment, the concentration of nanoparticles dispersed in the base structural can be between 0.01 wt % to 10 wt % g.

In one embodiment, the particles can be protein-based nanoparticle composites that can be responsive to a stimulus as described herein. The protein-based nanoparticle composites may be self-responsive to the stimulus or include a material described herein as being responsive to the stimulus. Such protein-based nanoparticle composites can be included in the base structural material of the scaffold.

### Kits

The invention also provides a kit. Such kits can be used for laboratory or for clinical applications. Such kits include a stimulus responsive component, e.g., a photoresponsive component described herein, and instructions for applying and irradiating the photoresponsive component to cross-link at least one protein reagent for laboratory use, or to bond, repair, or heal an animal tissue, e.g., a human tissue, particularly in a human patient. The kits can include a container for storage, e.g., a light-protected and/or refrigerated container for storage of the photoresponsive component. A photoresponsive component included in the kits can be provided in various forms, e.g., in powdered, lyophilized, crystal, or liquid form. Optionally, a kit can include an additional agent as described herein, e.g., a therapeutic agent, etc.

The kits described herein can also include a means to apply the scaffold to a tissue, for example, a syringe or syringe-like device, a dropper, a powder, an aerosol container, sponge applicator, and/or a bandage material. Kits can further include accessory tools for tissue approximation e.g. clips, standard weights, aspiration apparatus, and compression gauges.

Kits can include instructions for use, e.g., instructions for use in the absence of an exogenously supplied source of cross-linkable substrate, e.g., protein.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1: Laser Tissue Sealing for Nerve Anastomosis

A novel approach to seal and heal severed or damaged nerve tissue is described herein. In this approach, the sealant material, containing embedded nanoparticles is generated and characterized. Unmodified and chemically modified polypeptides and polymers (including hydrogels) are used as the sealant materials and different metallic nanoparticles including those from gold, silver, copper and aluminum are embedded in them. Laser light at different power densities and peak absorbance (corresponding to the maximal absorbance of the nanoparticles) are employed to generate temperatures in the range of 60-80° C. Ex vivo study follows with the sealant materials and operating conditions that result in temperatures of 60-80° C. Isolated rat sciatic nerves are severed and treated with the sealant materials at the different laser powers and the efficacy of sealing is investigated using tensile strength measurements. Chemical modifications (e.g. adhesion peptides) of the materials is carried out in order to facilitate greater sealing to the tissue. Those formulations that are effective are used in a model of sciatic nerve crush injury in vivo. Different bioactive molecules (e.g. growth factors) are delivered using the sealant material in order to facilitate rapid healing of the nerve tissue. A combination of the sealant materials, nanoparticles, and bioactives for nerve anastomosis and repair is the underlying invention.

The materials and methods employed in these experiments are now described.

Chitosan flakes (low molecular weight, >75% deacetylated), sodium carboxymethyl cellulose, and alginic acid sodium salt were purchased from Sigma-Aldrich. Silk was extracted from Bombyx mori cocoons based on a well-known protocol. Chitosan was dissolved in a 1.0 wt % aqueous acetic acid solution at a concentration of 2.0 wt %. Glutaraldehyde was diluted in 0.05% and was added to the chitosan solution in a 1 w/v %. Silk, cellulose, and alginate were dissolved in nano-pure water at the concentration of 2.0 wt %. 1 w/w % of synthesized gold or silver nanoparticles was added to the pre-gel solutions, then the polymeric solutions were cast in a glass bottom well plates at room temperature for 24 h. Then the cast films were rinsed nanopore water and dried at room temperature for 24 h to evaporate the solvent thoroughly and obtain the polymeric films.

A direct reduction method was used to synthesize silver nanoplates which showed a maximum absorbance at 800 nm. Briefly, in 19.272 mL of cold nanopure water, an aqueous solution of silver nitrate (0.05 M, 40 μL), trisodium citrate (75 mM, 0.4 mL), polyethylene glycol (PEG, Mw ˜3,500, 17.5 mM, 0.12 mL), and hydrogen peroxide (30 wt. %, 48 μL) were combined and vigorously stirred at room temperature in air. Sodium borohydride (NaBH₄, 100 mM, 120 μL) was rapidly injected into this mixture to obtain the nanoplates.

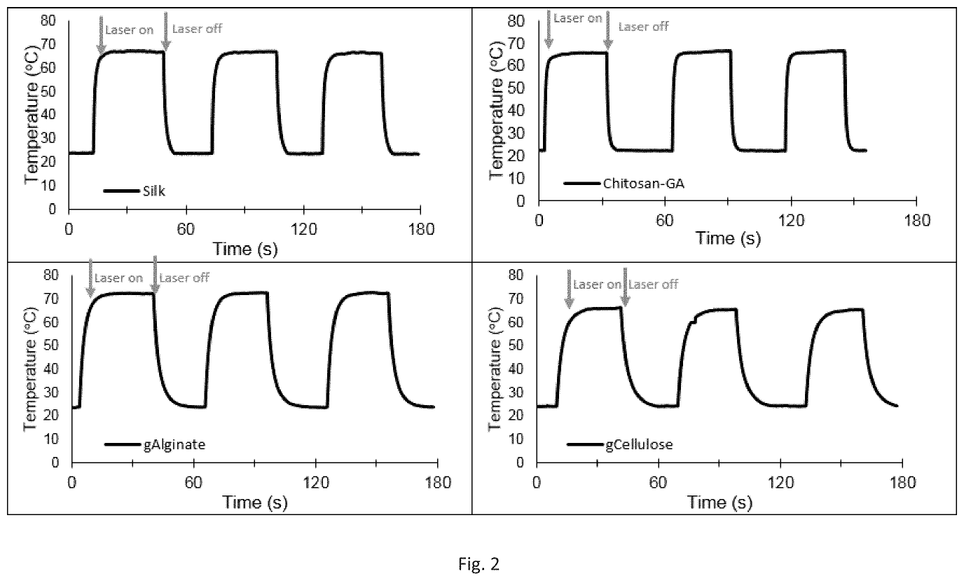

The photothermal response of the hydrogel containing nanoparticles was studied using a 5 W Ti:S infrared laser (Millenia) with wavelength tuned to 800 nm, corresponding to the maximum absorbance of the nanoparticles, was used at power densities of 1.59, 2.22, and 3.18 W/cm². In each power, the laser was on for 30 seconds and off for another 30 seconds. The cycle was repeated two more times. FIG. 2 shows temperature response upon applying laser beam. The film temperature reached up to 90° C. in the first 10 seconds after it is exposed to the laser. The nanoparticles embedded in the hydrogel could convert the laser light into thermal energy result in the temperature increase of the nanocomposite.

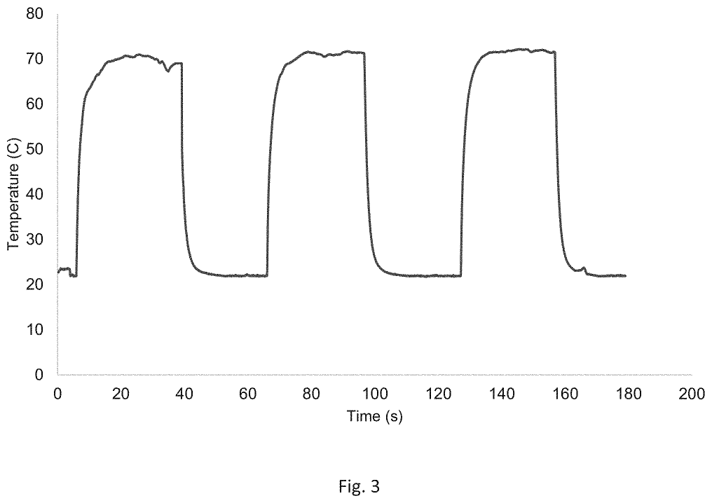

The films were tested to ex-vivo nerve tissue to seal the tow ends. The films were placed on the nerve tissue and were exposed to the near-IR laser beam corresponding to the maximum absorbance (800 nm) for a specific duration of time (1-5 minutes). The temperature was monitored during laser exposure. Elevation of the polymeric film and the tissue result in tissue-matrix integration and tissue sealing nerve FIG. 3.

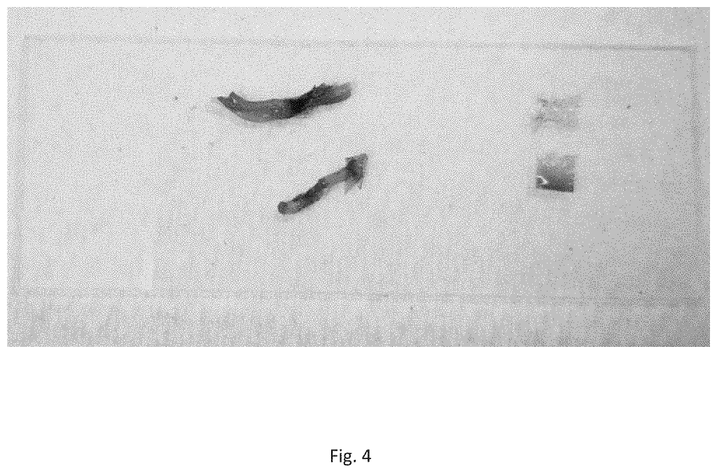

Nerve tissue was harvested from euthanized rat and cut in the middle. The hydrogel sample was placed over the top of the tissue in close proximity to the incision site and was subjected to the near-infrared laser at the power density of 1.47 for 3 minutes. The surface temperature which was recorded by IR camera was kept between 60-75° C. As shown in FIG. 4, the polymeric film triggered by laser sealed the incision.

### Example 2: Laser Activated Adhesives for Nerve Repair

Nerve damage from trauma affects over 350,000 patients annually in the U.S., including combat, sports injuries, and neuropathies, leading to loss of sensation, chronic pain, and even permanent disability. Suturing is the clinical standard for nerve apposition (approximation of connective tissues e.g. epineurium, the outer layer) but can cause inflammation, fibrosis, asymmetric tension, and is time-consuming for nerve surgeons; usually epineural suturing takes 2-6 hours. Glues and sealants have been explored as sutureless alternatives in nerve repair, but often suffer from inadequate and inconsistent nerve joining, rigidity, and cytotoxicity. Without adequate mechanical approximation, dehiscence or rupture occurs, and repeat surgery is often required. Reduction in procedure times, generation of minimal-tension approximation, and prevention of scar formation are critical for improving repair outcomes in peripheral nerve injuries.

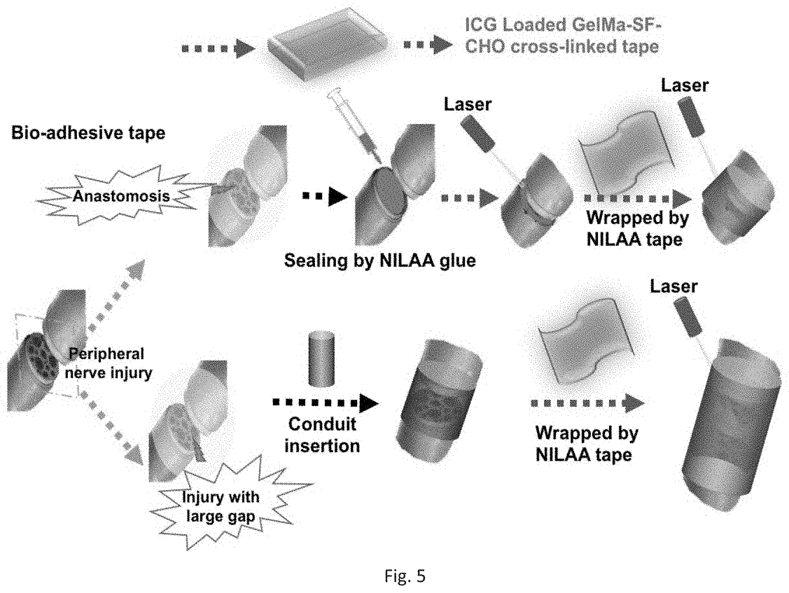

Near-infrared laser-activated adhesives (NILAAs) provide sutureless, fast, and precise tension-free nerve repair, and significant advantages of this approach is demonstrated for soft tissue (e.g. dermal) repair. As shown in FIG. 5 NILAAs can selectively absorb NIR, localize the heat into the adhesive and facilitate rapid end-to-end anastomosis and sealing of severed nerves. Further, NILAA tapes can provide fast, sutureless, and mechanically robust support and adhesion for grafting synthetic conduits to nerve stumps with large (>1 cm gaps).

Composites of gelatin methyacrylate (GelMa), silk fibroin (“silk”), and chitosan (CHO), embedded with an FDA-approved near-infrared absorbing dye indocyanine green (ICG) is developed as NILAA biomaterials for end-to-end anastomosis of nerves with small gaps (FIG. 5). GelMa within the composite is cross-linked using a photoinitiator in order to modulate the mechanical properties and stability of NILAAs. NILAA biomaterials is then characterized for their rheological, mechanical, and degradation properties. The temperature response of NILAAs to different NIR wavelengths and powers is determined and a mathematical model will be used to identify optimal conditions for nerve sealing. Ultimate tensile strength (UTS) and imaging of the NILAA-epineurium interface will be investigated following sealing in ex vivo nerve tissue.

The efficacy for NILAAs for nerve sealing and repair is determined using a transverse incision model and a crash model with a 10 mm gap in the sciatic nerve of Sprague Dawley rats. NILAAs are employed as glues to directly seal small incisions as wraps to seal and secure commercially available polylactic acid-co-caprolactone conduits (e.g. NEUROLAC), which is grafted into 1 cm sciatic nerve defects in rats. Sciatic functional index and muscle electromyographic (EMG) response is determined to investigate the efficacy of the NILAA approach compared to sutures and fibrin glue. Immunofluorescence, ELISA, histopathology and/or immunohistochemistry analyses is carried out to investigate inflammation, fibrosis, cell migration at the injury site in all cases. Histopathology, immunohistochemistry (IHC) and/or ELISA analyses is carried out for biomarkers including CD31, CD68, S-100, Knor26, etc. and inflammation biomarkers (TNF-α, IL-1β, IL-10), leading to insights into cellular and biochemical factors influencing nerve repair with NILAAs.

### Impact

NILAAs are an innovative approach for epineural sealing, repair and regeneration of small as well as large nerve defects leading to faster operation times and better quality of repair including low trauma, scarring, and inflammation, which make this technology highly attractive for clinical translation.

### Significance

Nerve damage from trauma including combat, accidents, sports injuries, and neuropathies, affects over 350,000 patients annually in the U.S., leading to loss of sensation, chronic pain, and even permanent disability. Crush and transection injuries form a significant bulk of peripheral nerve injuries and sciatic nerve is the most commonly affected among lower limb nerves. Every year, 50,000 nerve grafts are performed in the US and account for expenditures of $7 billion. Due to slow regeneration and limited repair in peripheral nerve injury (PNI), surgery is generally required for functional recovery. With current strategies, however, surgical repair for traumatic PNI has a 50% or lower likelihood of complete restoration of function, indicating an urgent need for approaches that can result in accelerated repair. Trauma-affected, nerve defects can be (1) small (<1 cm gap between nerve stumps), medium (1-3 cm gap), or large (>3 cm gap).

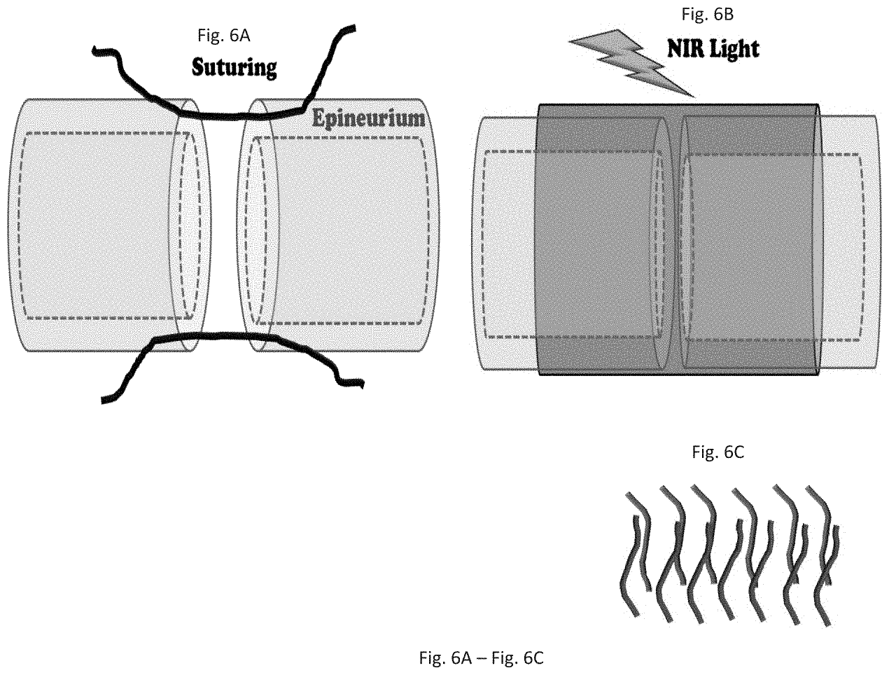

In nerve approximation, the surgeon aligns and connects the proximal and distal stumps directly. Suturing is the clinical standard for nerve repair and involves apposition of the outermost layer—epineurium—under a surgical microscope (FIG. 6A-FIG. 6C). Epineural suturing is time consuming, requires significant skill, and can result in chronic inflammation, fibrosis, and asymmetrical tension. Glues and sealants are being researched and developed as suture-less alternatives to avoid nerve tension and fibrosis. Conventional glues and sealants, however, often suffer from inadequate and inconsistent nerve joining, rigidity, brittleness, and toxicity. Without adequate mechanical approximation, gapping or rupture often occurs, and can lead to poor outcomes in patients.

Laser-assisted sealing is a promising sutureless approach for repair of tissues, including nerves. In this approach, an adhesive biomaterial absorbs laser light energy and converts it to heat. This is accompanied by a rise in local temperature which facilitates physical bonding of the sealant to the tissue, resulting in rapid sealing within a few seconds or minutes. Control of heat and use of effective adhesive biomaterials are key determinants of the success of laser sealing. To improve nerve repair outcomes over existing modalities, it is necessary to reduce operative times, use appropriate biomaterials, create tension-free support, and prevent scar formation.

In this study, a novel near-infrared laser-activated adhesives (NILAAs) is developed to overcome the limitations of the current standard of care, particularly with suturing for nerve repair, leading to promising outcomes. NILAAs integrate with the epineurium (thickness in sciatic nerve ˜120 mm; FIG. 6A-FIG. 6C). The near-infrared laser-activated adhesives (NILAAs) of this study provide sutureless, fast, and precise tension-free nerve repair.

The materials and methods employed in these experiments are now described.

### Generation and Characterization of NILAAs for Nerve Repair

Composites of GelMa/silk fibroin (“Silk”)/high molecular weight chitosan (GM/silk/CHO) hybrid solutions containing 10 w/v % GelMa and different amounts of CHO (0.5-2%) and silk (2-10%) are generated by mixing their aqueous solutions at 40° C. The composite solutions are transferred to 5 mL syringe and are stored at 4° C. as NILAA glues. To prepare tapes, 0.05 mM eosin Y, 225 mM triethanolamine (TEA) and 74 mM 1-vinyl-2 pyrrolidinone (NVP) are added to the composite solutions and a film is cast on plastic coverslips (20×20 mm and ˜200 μm thickness. The films are then exposed to visible light (500 lumens) for 10 min to cross-link GelMa. Films are dried at room temperature overnight.

Mechanical properties, tensile strength, absolute shear modulus (|G*|) and loss angle (δ), which indicate stiffness and internal energy dissipation under dynamic loading, respectively, are determined for all NILAAs using ASTM standards (e.g. D30309/D3039M). The storage (elastic) modulus G′ and loss (viscous) modulus G″ versus temperature are measured at a gap of 0.105 mm and a frequency of 1.0 Hz. Interdigitation between NILAAs and the epineurium occurs between 60-65° C. and the temperature response study help optimize conditions for effective tissue sealing and minimal thermal damage. Temperature profiles of NILAAs (containing 0.1 mM ICG) activated by continuous-wave 808 nm NIR hand-held lasers at 450 mW/cm² is determined using a digital infrared camera; corresponding composites without ICG are used as controls. Morphology and surface properties of NILAAs are evaluated using scanning electron microscopy (SEM). Swelling and degradation rates are measured by monitoring the volume change and weight loss during the time, respectively, in PBS.

### Biomechanical Properties of NILAAs for Nerve Sealing Ex Vivo

Up to five different formulations that demonstrate high elasticity and tensile strength, low swelling and quick temperature response to laser irradiation are investigated for nerve sealing ex vivo. Laser conditions (power, wavelength and exposure time) are modulated in order to identify the conditions that result in the highest adhesion and tensile strength recovery following NILAA sealing. A quantitative tensile strength test (ASTM F2258-05) is employed to determine the efficacy of the seal using methods similar to those in the previous studies. Ultimate tensile strength (UTS) and strain at UTS of the sealed nerve is measured for the NILAA glue group in comparison to suture and fibrin groups as control. Based on these adhesion studies, the most optimal NILAA formulation and laser conditions for in vivo studies is identified.

### Interface Visualization and Thermal Modeling

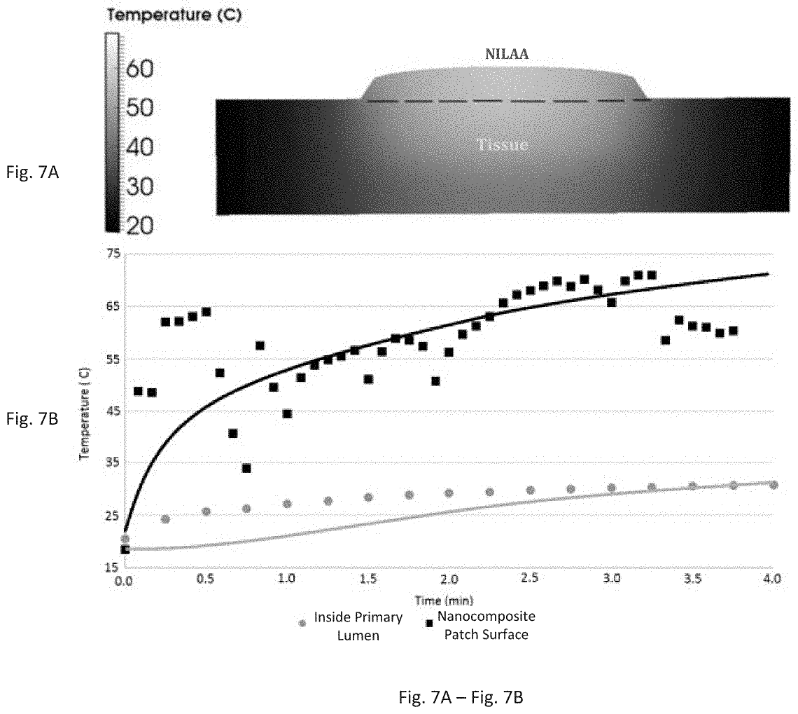

Laser sealing is a rapid procedure in which the light is moved from one location to another in the wound area over several passes; for example, 4-6 passes are made over a 1 cm incision. Thus, the risk of thermal damage of tissue is largely eliminated because of low residence time at any location; indeed, no sign of discomfort is seen in mice undergoing dermal laser sealing. In order to identify optimal conditions, a mathematical model is developed for temperature responses in nerves. Details of the modeling, including equations and parameters used, are provided in a recent publication. From this study on sealing intestinal incisions using collagen-gold nanorod NILAAs, it was learnt that the temperature rise is largely confined to the region immediately below the NILAA (FIG. 7A-FIG. 7B). This study utilized a continuous laser treatment for 4-8 minutes at a given location, which is an exaggerated case for laser sealing surgical procedures due to the operation described above; laser irradiation is only for a few seconds at a given location due to continuous movement. The model is modified to reflect the laser application methodology used in actual procedures, and dimensions and parameters specific to nerves are obtained from the literature. The heat transfer model is based on a modified Pennes bioheat equation coupled with laser operating conditions (energy). An Arrhenius cell death module is employed in concert with the heat transfer equation to predict cell death for different laser operating conditions and NILAA properties. Model predictions for temperature is confirmed with infrared (thermal) imaging as in the previous studies. In concert with imaging (below), laser conditions and operating procedures that restrict the temperature rise to the epineurium (depth of 50-100 mm) are identified in order to minimize thermal damage to the nerve.

NILAAs are conjugated with Alexa Fluor 594 dye and upon laser sealing, ex vivo nerve tissue are sectioned into thin sections (˜10-20 mm thickness) using a microtome, fixed and counterstained with green-fluorescent dye-conjugated primary (or secondary) antibodies to tissue collagen I and collagen IV. Confocal fluorescence microscopy) is employed to visualize the integration of NILAAs with native tissue proteins. Sectioning and concomitant microscopy enables estimation of the depth of NILAAs penetration in the tissue (epineurium); which the depth of the NILAA-epineurium interface is anticipated to be ˜25-50 mm.

In case of unexpected complications and/or sub-optimal sealing performance with GelMa/silk/CHO NILAAs, silk and collagen based NILAAs are evaluated. In case of sub-optimal mechanical properties, cross linking of silk and chemical modification approaches are investigated in order to improve tissue adhesion if necessary.

### Evaluation of NILAA Glues and Tapes for End-to-End Nerve Anastomosis of Small and Sealing Regenerative Conduits in Large Nerve Gaps

Small defects can be approximated by approximating the nerve stumps directly and regeneration-promoting conduits are efficient devices for repairing nerve injuries with large gaps. In both cases, suturing is used to approximate nerve stumps. The objective of this work is to develop a NILAA-based sutureless approach that provides better a biomechanical performance of sealing as well as quality of repair and regeneration.

### General Procedure

Adult male Sprague Dawley rats, 300-400 g, are housed in the animal BSL-2 facility at ASU and acclimated for one week. Rats are anesthetized, and the left hind legs are shaved, and residual hair removed using hair removal cream. Aseptic techniques are employed to disinfect the skin (i.e., application of isopropyl alcohol or betadine) are used to ensure sterility. After skin incision and dissection of the muscle planes, the sciatic nerve are identified and isolated. Connective tissue surrounding the nerve are gently removed using iris micro scissors at least 1 cm distal from the trifurcation point.

### In Vivo Evaluation of NILAAs for Small Nerve Defects

The sciatic nerve form left hind leg is incised along the diameter for end-to-end approximation (right hind leg will be used as a control). Animal numbers, based on published tensile strength values for silk-GNR NILAAs for dermal repair in mice, indicate 8 mice/group for biomechanical recovery studies. The same number is used for sciatic nerve model in rats for the following groups: 1. Intact nerve—sham surgery group (no approximation), 2. Incised—NILAA glue, 3. Incised—suture (control) and 4. Incised—fibrin glue (control) 5. Incised nerve—Suture+secured by NILAA glue (combination) 6. Intact nerve—NILAA glue. These numbers were calculated using a t-test, difference between two independent means, two-tailed alpha 0.05, power 80%, effect size 1.70, t-crit 2.17, df 12 indicate n=8/group. Therefore 6 groups*8 rats/group=48 rats is needed for this studies. In addition, 4 rats/group is needed for histopathology and 4 rats/group for ELISA analyses (described in following sections), indicating 8*6=48 rats. Total rats needed: 48+48=96+—10% extra for incidentals=106 rats. Studies are carried over a 12-week timeframe. Laser conditions optimized above is employed.

### In Vivo Evaluation of NILAAs for Securing Regenerative Conduits for Large (1 cm) Defects

A 1 cm section of the sciatic nerve is severed in order to mimic larger defects in peripheral nerve injuries. A commercially available nerve regeneration conduit (e.g. NEUROLAC) is inserted between the distal and proximal stumps and secured using the following (1) sutures (2) fibrin glue (3) NILAA tape (wrap)+laser (4) Suture+NILAA wrap. Two additional groups—sham surgery (intact nerve, no approximation device) and intact nerve covered with NILAA and lasered—are investigated as in the above section, leading to a total of 6 groups. Similar to the incision model, a total of 106 rats is required, bringing the total to 212 rats in this study. As before, the studies are carried out over a 12 week timeframe.

### Recovery of Neural Function

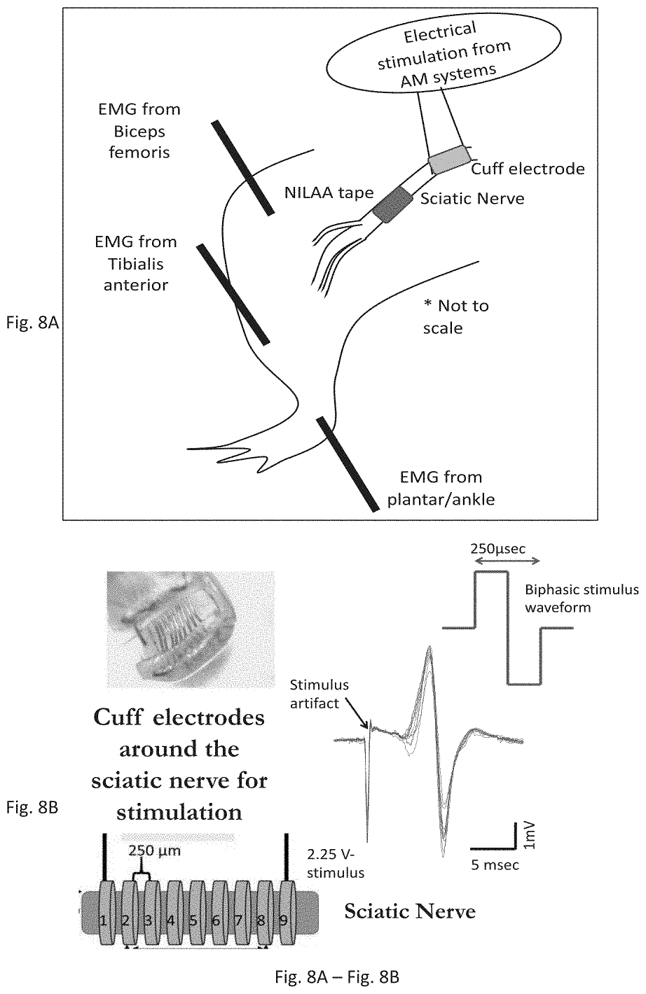

In these rodent experiments, nerve cuff electrodes are used (FIG. 8A—FIG. 8B) to stimulate the sciatic nerve with biphasic rectangular voltage pulses (250 μs duration, 10 Hz repetition rate) and record evoked muscle electromyographic (EMG) responses using needle electrodes placed downstream in 3 different muscle groups (Biceps femoris, Tibialis anterior and plantar/ankle). The incision site approximated using NILAA, sutures, etc. on the sciatic nerve, is located between the nerve cuff electrodes and the EMG electrodes (FIG. 8A-FIG. 8B). These electrodes are placed during the time of surgeries and the method have been demonstrated in a recent publication. Briefly, custom fabricated nerve cuff electrodes (Microprobes Inc., Gaithersburg, Md., USA) are surgically implanted with 9 rings of 100 μm diameter platinum electrodes spaced 250 μm apart as shown in FIG. 8A and FIG. 8B. The total distance between the inner edge of electrode rings ‘1’ and ‘9’ is ˜2.7 mm. Each ring on the cuff has an impedance of ˜2 kΩ at 1 kHz. The cuff electrodes are stimulated using AM systems neurostimulating system (model 2100 isolated pulse stimulator). The nerve cuff is placed approximately 1 cm distal from the trifurcation point where the sural, peroneal and tibial bundles split. The cuff is placed such that the insulating silicone bottom under the rings is the only contact point with the rat body to ensure no contact with surrounding muscle groups to prevent potential off-target stimulation effects. Leads from the cuff electrodes is routed transcutaneously to external connectors sutured on the rat hind limb. All incisions are sutured after implantation and the surgical sites are monitored constantly for any signs of infection. Needle-based electromyography (EMG) is used to measure evoked responses from different muscle groups in the hind limb. Disposable monopolar needle electrodes (Rhythmlink™, Columbia, S.C., USA) are placed in digit 5 of the rat ipsilateral hind leg paw. The animal are grounded with a needle electrode in the opposite hind leg. EMGs are recorded using Intan™ recording system (Intan, Los Angeles, Calif., USA) and analyzed in MATLAB′ offline. The recordings are digitally filtered on the Intan™ system using a bandpass filter from 100-3000 Hz to remove motion artifacts. EMG recordings are analyzed for 10 repetition trials of each stimulation condition. Evoked muscle EMG responses to different stimulation voltages are measured from the 3 muscle groups periodically every 3 days till the end of study (12 weeks). These recordings are carried out for all groups listed above in order to track functional restoration in every case. Sensory and motor function profile after nerve repair are also evaluated every week for 12 weeks post-surgery using pinch test and Walking-Track Analyses (sciatic nerve function index). After 12 weeks, rats are euthanized, wet muscle mass is measured, and the anastomotic site is harvested for biochemical analyses.

### Biomechanical Recovery

For all groups, ultimate tensile strength, strain, and resilience is determined using a TA.XT instrument and using methods similar to the published results with other tissues.

### Cellular and Biochemical Analyses

The anastomotic site (including the incisions and the conduit, extended ˜5 mm from each proximal and distal nerve stumps) is harvested for histological assessment and is fixed in 10% neutral buffered formalin. All samples designated for protein extraction are stored at −80° C. For quantification of macrophage invasion of the repair site and determine their specific phenotype, histopathology, and immunohistochemistry (IHC) analyses are carried out for specific biomarkers including CD68 (M1 marker), and CD206 (M2 marker), GAP43 (regeneration marker), S-100 for mature Schwann cells, Oct6 for immature Schwann cells, and Krox20 for myelinating Schwann cells in order to investigate extent of repair and regeneration. Samples are processed at ASU and given to Mayo Clinic for reading and interpretation; where they are blinded to the sample information. Biomarkers for inflammation (TNF-α, IL-1β, IL-10) is quantified using ELISA. Total collagen is assessed to evaluate healing using Masson's trichome staining and collagen I, III, and IV deposition along with TGF-b is analyzed to determine potential for scaring in tissue.

In case of unsatisfactory performance, tape is chemically modified to facilitate the formation of a covalent bond at the interface of both synthetic conduit and biological materials for better adhesion; for example, photochemical bonding with Rose Bengal dye is explored in combination with laser sealing. Mechanical recovery of the system can be improved by changing the composition of NILAAs using chitosan with different molecular weights and degree of deacetylation or replacing chitosan with chitosan-methacrylate to further strengthen bonding. In case regeneration is not satisfactory, the delivery of growth factors including BDNF or SDF is explored in order to accelerate regeneration. NILAAs can indeed be used as depots for drug delivery.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method for welding tissue wounds in a subject, wherein the method comprises the steps of:
providing a scaffold, wherein the scaffold comprises a base structural material and at least one stimulus responsive component;
aligning a first edge of the wound with a second edge of the wound;
placing the scaffold over or in between the first edge of the wound and the second edge of the wound; and
exposing the scaffold to an internal or external energy source, wherein the stimulus responsive component absorbs the energy and subsequently generate heat and causes the first edge of the wound and the second edge of the wound to adhere to each other and/or to the scaffolds.

2. The method of claim 1, wherein the structural material is selected from the group consisting of: a natural polymer, a synthetic polymer, and combinations thereof.

3. The method of claim 1, wherein the scaffold is selected from the group consisting of: a polymeric matrix, a hydrogel, a film, an electrospun scaffold, a tubular conduit, and combinations thereof.

4. The method of claim 2, wherein the structural material is chemically modified to facilitate adhesion of the scaffold to the tissue.

5. The method of claim 1, wherein the stimulus responsive material is selected from the group consisting of: a photoresponsive material, a magnetic responsive material, an electrically responsive material, a chemically responsive material, and combinations thereof.

6. The method of claim 4, wherein the stimulus responsive material is a photoresponsive material.

7. The method of claim 5, wherein the stimulus responsive material is in particle form.

8. The method of claim 6, wherein the stimulus responsive material is selected from a group consisting of gold nanorods, gold nanostars, gold nanoparticles, gold nanospheres, gold nanostars, indocyanin green, neodymium-doped nanoparticles, carbon nanotubes, organic nanoparticles, alumina nanoparticles, copper nanoparticles or near-infrared absorbing dyes, silver nanoplats/prisms, silver nanoparticles, and combinations thereof.

9. The method of claim 5, wherein the photoresponsive material is stimulated with a laser.

10. The method of claim 8, wherein the laser wavelength is in a range of between 800 nm to about 2700 nm.

11. The method of claim 8, wherein the laser is delivered in pulse mode wherein a series of short pulses are applied.

12. The method of claim 8, wherein the laser is delivered in a continuous mode.

13. The method of claim 1, wherein the scaffold further comprises an active agent selected from the group consisting of: an anti-inflammatory, a wound healing agent, a growth factor, and combinations thereof.

14. The method of claim 1, wherein the structural material is biodegradable.

15. The method of claim 1, wherein the tissue is selected from a group consisting of: skin, mucosal tissue, bone, blood vessels, neural tissue, hepatic tissue, pancreatic tissue, splenic tissue, renal tissue, bronchial tissue, tissues of the respiratory tract, tissues of the urinary tract, tissues of the gastrointestinal tract, tissues of the gynecologic tract, and combinations thereof.

16. The method of claim 14, wherein the tissue is a neural tissue.

17. A composition for welding tissue wounds in a subject, wherein the composition comprises a scaffold, wherein the scaffold comprises a base structural material and at least one stimulus responsive component.

18. The composition of claim 18, wherein the scaffold is selected from the group consisting of: a polymeric matrix, a hydrogel, a film, an electrospun scaffold, a tubular conduit, and combinations thereof.

19. The composition of claim 18, wherein the structural material is selected from the group consisting of: a natural polymer, a synthetic polymer, and combinations thereof.

20. The composition of claim 18, wherein the stimulus responsive material is selected from the group consisting of: a photoresponsive material, a magnetic responsive material, an electrically responsive material, a chemically responsive material, and combinations thereof.
